(19) United States
(12) Patent Application Publication
(10) Pub. No.: US 2006/0211993 A1
(43) Pub. Date: Sep. 21, 2006

(54) Impact resistant conduit

(76) Inventors: Derek Andrews, Gloucestershire (GB); Travis Steel, Gloucestershire (GB)

Correspondence Address: YOUNG & THOMPSON, 745 SOUTH 23RD STREET, 2ND FLOOR, ARLINGTON, VA, 22202, US

(21) Appl. No.: 11/358,127

(22) Filed: Feb. 22, 2006

(30) Foreign Application Priority Data
Feb. 24, 2005 (GB) 0503774.2

(51) Int. Cl. A61M 5/00

(52) U.S. Cl. 604/171

(57) ABSTRACT

An impact resistant conduit for electrical wiring, the conduit being formed from impact resistant material comprising unidirectionally braided elongate flexible elements which are consolidated to form a homogenous matrix. The elongate flexible elements include a composite mixture of resin and carbon fibre. The carbon fibre is impregnated with the resin.

### BACKGROUND OF THE INVENTION

The present invention relates to an impact resistant conduit, particularly, but not exclusively, for use with electrical wiring.

Exposed electrical wiring, for example forming part of a wiring harness, is presently protected from damage by being housed or sheathed in metal tubing. A typical metal is stainless steel.

However, metal is relatively heavy and, in the case of stainless steel, extremely expensive.

The present invention seeks to overcome this problem.

### SUMMARY OF THE INVENTION

In accordance with a first aspect of the present invention, there is provided an impact resistant conduit for electrical wiring, the conduit being formed from impact resistant material comprising unidirectionally braided elongate flexible elements which are consolidated to form a homogenous matrix, the elongate flexible elements including a composite mixture of resin and carbon fibre, the carbon fibre being impregnated with the resin.

In accordance with a second aspect of the invention, there is provided a method of forming an impact resistant conduit, in accordance with the first aspect of the invention, the method comprising the steps of: (a) unidirectionally braiding a plurality of said elongate flexible elements onto a former to form flexible braided material; (b) heating the flexible braided material so that the plurality of elongate elements form a homogeneous matrix, and thus form a unitary impact resistant conduit; and (c) forming the unitary impact resistant conduit into a desired shape.

The invention will now be more particularly described, by way of example only, with reference to the accompanying drawings.

### BRIEF DESCRIPTION OF THE DRAWINGS

FIG. 1 shows part of apparatus used in a method of forming an impact resistant tube, in accordance with the second aspect of the invention; and

FIG. 2 shows a jig used in the method of forming an impact resistant tube.

### DESCRIPTION OF THE PREFERRED EMBODIMENT

One embodiment of an impact resistant tube will now be described, by way of example only, with reference to the drawings. The impact resistant tube, having an uninterrupted circumference, is formed from impact resistant thermoplastic material being a composite mixture of carbon fibre, poly-paraphenylene terephthalamide-based (for example Kevlar®) fibre, and resin.

This material is impregnated with the resin, and has been available, although not in the form of elongate flexible elements, from Schappe Ltd. at B.P. 8901800 Charnoz, France under the trade name TPFL®.

The resin is one or more of polyetheretherkeyton (PEEK), polyphenylenesulphide (PPS), polyetherimide (PEI), polyamide (PA) polypropylene (PP); and the fibres are provided in an amount of 40% to 70% fibre volume fraction. No halogens are present.

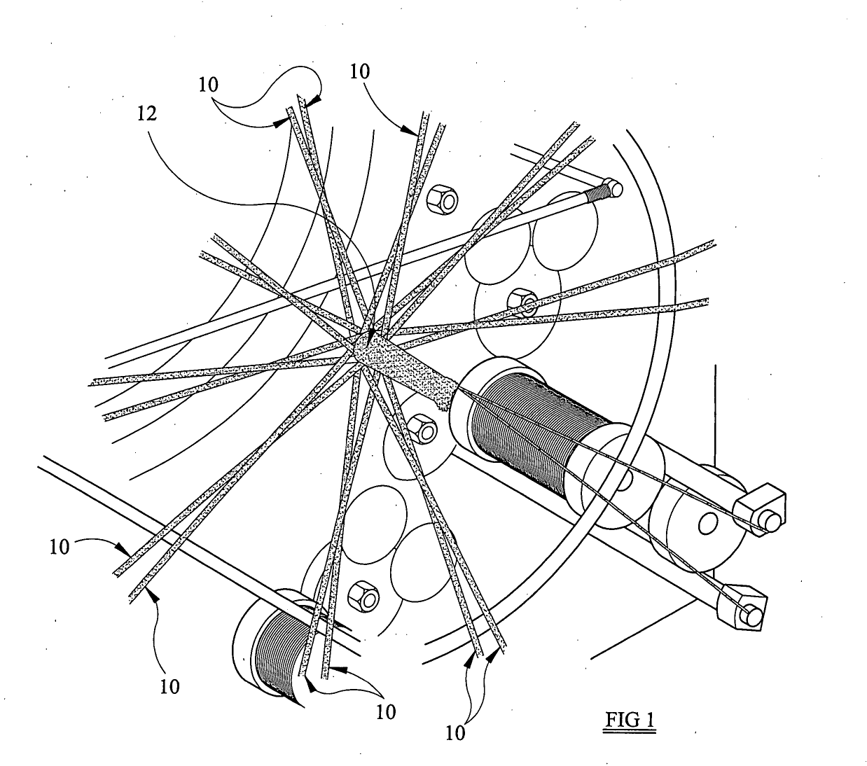

The impact resistant material is initially formed as an elongate flexible element, for example a yarn, tape or thread. Referring to FIG. 1, a plurality of the elongate elements 10 is braided unidirectionally. The braiding is initiated, and then a rigid elongate former (not shown) is coaxially inserted, and braiding is completed on the former.

The flexible braided material 12 and former are placed in an oven and heat treated, so that the elongate elements form a homogenous matrix or unitary tube on the former.

The unitary tube is cooled, and the former is removed. The unitary tube is then post-heated to slightly soften the impact resistant material to allow post-forming.

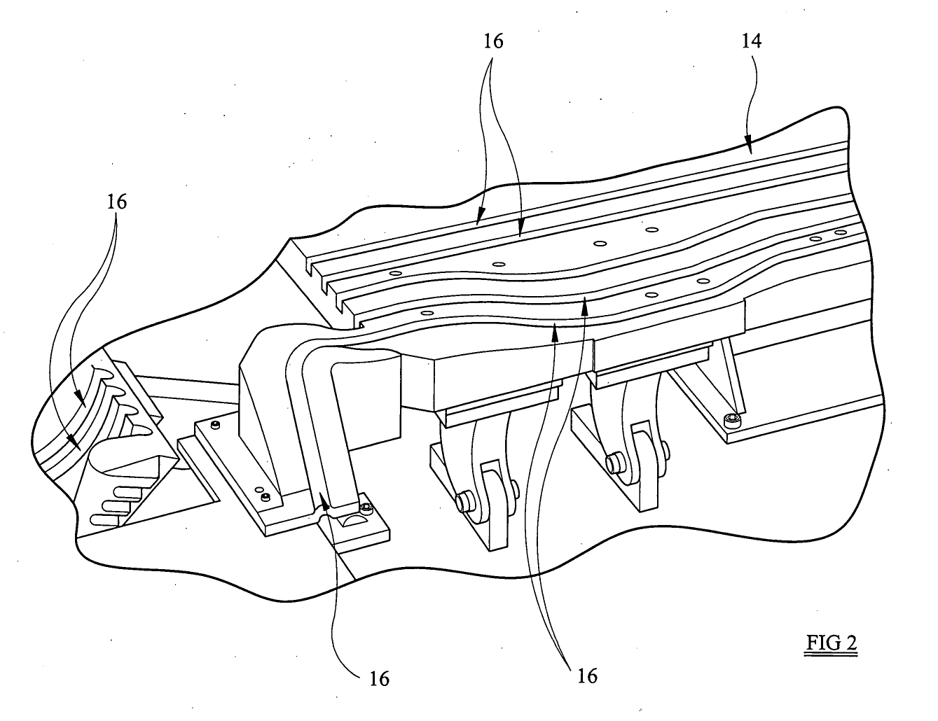

As shown in FIG. 2, a jig 14 is utilised to post-form the unitary tube. Once softened, the unitary tube is placed in a channel 16 of the jig corresponding to a desired shape of impact resistant tube. The unitary tube is held in the jig 14 until the material has cooled. Once cooled, the impact resistant tube assumes a rigid hollow structure which retains its shape.

In a second embodiment of a method for forming the impact resistant tube, unidirectional braiding of a plurality of the elongate elements is again initiated. A former is again coaxially inserted. However, the former is an inflatable and deflatable bladder-type former. Unidirectional braiding is completed on the deflated former.

The flexible braided material and deflated former are placed in a mould which defines the finished shape of the impact resistant tube. The former is inflated, so that the braided material is pressed against the internal sides of the mould. The mould together with the braided material and inflated former are heated in an oven. The yarns or threads again form a homogenous matrix or unitary tube along the former.

The mould is cooled, and the former is deflated and removed from the unitary tube. The impact resistant conduit is thus formed and shaped simultaneously, or substantially simultaneously, and without the need for a separate jig.

One or both formers, and thus the finished tube, can have a round, oval or substantially quadrilateral cross-section. Other cross-sectional shapes are possible.

The tube typically has diameters which range from 6 mm to 50 mm, and with a wall thickness that ranges from 0.3 mm to 5.0 mm.

Shapes such as two dimensional and three dimensional bends, flairs and belling can be imparted to the tube by the methods described above without reducing structural integrity. Consequently, complex and tortuous paths can be formed without weakening the tube.

Once cured, the tube maintains structural integrity up to 65% crush resistance; is 75% to 80% lighter by volume than stainless steel; and is heat resistant in the range of −150° C. to 350° C., though more preferable operating parameters are −70° C. to 260° C.

The tube exhibits excellent flame, smoke and toxicity resistance as well as being resilient to fuel, oil, solvent and chemical exposure.

An interior coating can be applied to the former before winding the impact resistant material. The coating can be a abrasion reducing coating, such as PTFE; an electrical and/or EMC screening coating; and/or a fluid impermeable coating. More specifically, the fluid impermeable coating can be a liquid impermeable coating.

An exterior coating may alternatively, or additionally, be provided. The coating can also be an abrasion resistant lining, such as PTFE. Alternatively, the coating can be plating, which easily takes to the impact resistant material. The plating can be, for example, gold, nickel, copper, zinc or any other plating suitable for a given application.

The exterior coating can also provide electrical and/or EMC screening.

The tube can be integrated as part of a Connector adaptor, Backshell, harness boot; and/or primary conduit. This is particularly beneficial in protecting an exposed electrical harness on, for example, the undercarriage of an aeroplane which is especially vulnerable to projectile impact during take-off and landing. Furthermore, due to the light weight of the tube, overall weight is reduced in comparison to traditional metal impact resistant tubing.

The impact resistant tube is stiff, reducing the need for brackets and ‘p’ clips supporting the wiring or harness.

The tube can also be utilised, for example, in the masts of ships due to its high rigidity and low weight.

In use, electrical wiring may be fully enclosed by the impact resistant tube, or may enter and exit the tube as required.

Although the impact resistant tube has been described as being applicable to aircraft and ships, it can be utilised on any transport, or indeed in any area, where exposed electrical wiring is subject to projectile damage.

Other types of conduit, as alternatives to a tube, can be formed from the impact resistant material. The conduit may have a non-continuous circumference, for example having a U- or C-shaped cross-section.

Although Kevlar® fibres are used in conjunction with the carbon fibres, it may be possible to dispense with the Kevlar® fibres, or utilise alternative fibres, such as R-Glass.

It is thus possible to provide an impact resistant conduit which is more cost-effective to produce than a traditional stainless steel protective tube or conduit, which exhibits better impact resistance, and which is lighter.

The embodiment described above is given by way of example only, and various other modifications will be apparent to persons skilled in the art without departing from the scope of the invention, as defined by the appended claims.

What is claimed is:

1. An impact resistant conduit for electrical wiring, the conduit being formed from impact resistant material comprising:
unidirectionally braided elongate flexible elements which are consolidated to form a homogenous matrix,
the elongate flexible elements including a composite mixture of resin and carbon fibre, and
the carbon fibre being impregnated with the resin.

2. An impact resistant conduit as claimed in claim 1, wherein the conduit is a hollow tube having an uninterrupted circumference.

3. An impact resistant conduit as claimed in claim 1, wherein the resin of the impact resistant material is polyetheretherkeyton (PEEK), polyphenylenesulphide (PPS), polyetherimide (PEI), polyamide (PA), polypropylene (PP), or a combination thereof.

4. An impact resistant conduit as claimed in claim 1, wherein the said material further comprises poly-paraphenylene terephthalamide-based fibre.

5. An impact resistant conduit as claimed in claim 1, wherein the or both kinds of said fibres of the said material are in an amount of 40% to 70% fibre volume fraction.

6. An impact resistant conduit as claimed in claim 1, wherein the conduit is 65% crush resistant.

7. An impact resistant conduit as claimed in claim 1, wherein the conduit is 25% lighter than stainless steel by volume.

8. An impact resistant conduit as claimed in claim 1, wherein the conduit is heat resistant to in the range of −150° C. to 350° C.

9. An impact resistant conduit as claimed in claim 8, wherein the conduit is heat resistant in the range of −70° C. to 260° C.

10. An impact resistant conduit as claimed in claim 1, wherein the tube is shape formable.

11. An impact resistant conduit as claimed in claim 1, further comprising an interior coating and/or an exterior coating.

12. An impact resistant conduit as claimed in claim 11, wherein the interior coating is an abrasion reducing coating; an electrical and/or EMC screening coating; and/or a fluid impermeable coating.

13. An impact resistant conduit as claimed in claim 11, wherein the exterior coating is plating.

14. An impact resistant conduit as claimed in claim 11, wherein the exterior coating provides electrical and/or EMC screening.

15. A method of forming an impact resistant conduit as claimed in claim 1, comprising the steps of:
a. unidirectionally braiding a plurality of said elongate flexible elements onto a former to form flexible braided material;
b. heating the flexible braided material so that the plurality of elongate elements form a homogeneous matrix, and thus form a unitary impact resistant conduit; and
c. forming the unitary impact resistant conduit into a desired shape.

16. A method as claimed in claim 15, wherein the former is a rigid former, and further comprising a step (d), between steps (b) and (c), of cooling the unitary conduit, removing the rigid former, and then post-heating the unitary conduit.

17. A method as claimed in claim 15, wherein the former is an inflatable and deflatable former, and, in step (b), the braided impact resistant material and former are positioned in a mould, so that steps (b) and (c) occur simultaneously or substantially simultaneously.

18. A method as claimed in claim 17, wherein, in step (a), the former is deflated, and in steps (b) and (c), the former is inflated.
